SEQUENCE LISTING (148 sequences)

SEQ ID NO: 1 - DNA, 33 bp, Homo sapiens
tcggtcagaggctagctacaacgatgcgttgct

SEQ ID NO: 2 - DNA, 33 bp, Homo sapiens
ggcgtacgaggctagctacaacgactgctcggt

SEQ ID NO: 3 - DNA, 33 bp, Homo sapiens
ggcggcgtaggctagctacaacgagacctgctc

SEQ ID NO: 4 - DNA, 33 bp, Homo sapiens
ctcgggtcaggctagctacaacgactgggtagc

SEQ ID NO: 5 - DNA, 33 bp, Homo sapiens
tcctctgcaggctagctacaacgacggggtcct

SEQ ID NO: 6 - DNA, 33 bp, Homo sapiens
actctgcaaggctagctacaacgatctgcgagc

SEQ ID NO: 7 - DNA, 33 bp, Homo sapiens
gggcgacgaggctagctacaacgatctgcaatt

SEQ ID NO: 8 - DNA, 33 bp, Homo sapiens
aaggggcgaggctagctacaacgagactctgca

SEQ ID NO: 9 - DNA, 33 bp, Homo sapiens
aaaacgggaggctagctacaacgacaggttgta

SEQ ID NO: 10 - DNA, 33 bp, Homo sapiens
agaataaaaggctagctacaacgagggaccagg

SEQ ID NO: 11 - DNA, 33 bp, Homo sapiens
atggcagaaggctagctacaacgaaaaacggga

SEQ ID NO: 12 - DNA, 33 bp, Homo sapiens
aactgggtaggctagctacaacgaggcagaata

SEQ ID NO: 13 - DNA, 33 bp, Homo sapiens
atccaaaaaggctagctacaacgatgggtatgg

SEQ ID NO: 14 - DNA, 33 bp, Homo sapiens
aggggaagaggctagctacaacgaaaaaatcca

SEQ ID NO: 15 - DNA, 33 bp, Homo sapiens
ttttaaaaaggctagctacaacgatatcttgga

SEQ ID NO: 16 - DNA, 33 bp, Homo sapiens
gtggggggaggctagctacaacgagggaaggct

SEQ ID NO: 17 - DNA, 33 bp, Homo sapiens
gttgaatgaggctagctacaacgattgctttcg

SEQ ID NO: 18 - DNA, 33 bp, Homo sapiens
gtcgttgaaggctagctacaacgagatttgctt

SEQ ID NO: 19 - DNA, 33 bp, Homo sapiens
ggcccggaaggctagctacaacgaccgcgcgcg

SEQ ID NO: 20 - DNA, 33 bp, Homo sapiens
tcacctccaggctagctacaacgaggcctcggc

SEQ ID NO: 21 - DNA, 33 bp, Homo sapiens
ccgccgtcaggctagctacaacgactccatggc

SEQ ID NO: 22 - DNA, 33 bp, Homo sapiens
ggtggctcaggctagctacaacgaccagcgcgg

SEQ ID NO: 23 - DNA, 33 bp, Homo sapiens
cgttgagcaggctagctacaacgaggcggggtg

SEQ ID NO: 24 - DNA, 33 bp, Homo sapiens
ccgcgtccaggctagctacaacgagtaggagtg

SEQ ID NO: 25 - DNA, 33 bp, Homo sapiens
cagcgggtaggctagctacaacgatgcgccgcg

SEQ ID NO: 26 - DNA, 33 bp, Homo sapiens
gcacatccaggctagctacaacgactcctccgg

SEQ ID NO: 27 - DNA, 33 bp, Homo sapiens
aaaagcacaggctagctacaacgaccacctcct

SEQ ID NO: 28 - DNA, 33 bp, Homo sapiens
taaaaagcaggctagctacaacgaatccacctc

SEQ ID NO: 29 - DNA, 33 bp, Homo sapiens
gaccgtcgaggctagctacaacgagttaaaaag

SEQ ID NO: 30 - DNA, 33 bp, Homo sapiens
ttgccttgaggctagctacaacgacgtcgatgt

SEQ ID NO: 31 - DNA, 33 bp, Homo sapiens
agggcgggaggctagctacaacgagtggttgcc

SEQ ID NO: 32 - DNA, 33 bp, Homo sapiens
tggccctgaggctagctacaacgacgagtttcc

SEQ ID NO: 33 - DNA, 33 bp, Homo sapiens
acctctgcaggctagctacaacgacgtggccct

SEQ ID NO: 34 - DNA, 33 bp, Homo sapiens
cggagggtaggctagctacaacgactctgcacc

SEQ ID NO: 35 - DNA, 33 bp, Homo sapiens
ggcggcacaggctagctacaacgactggctccc

SEQ ID NO: 36 - DNA, 33 bp, Homo sapiens
cgggcggcaggctagctacaacgaacctggctc

SEQ ID NO: 37 - DNA, 33 bp, Homo sapiens
agggatccaggctagctacaacgagaagcagag

SEQ ID NO: 38 - DNA, 33 bp, Homo sapiens
gggtagggaggctagctacaacgaccatgaagc

SEQ ID NO: 39 - DNA, 33 bp, Homo sapiens
gggctgagaggctagctacaacgatccaggggg

SEQ ID NO: 40 - DNA, 33 bp, Homo sapiens
gtggatggaggctagctacaacgagtcttggag

SEQ ID NO: 41 - DNA, 33 bp, Homo sapiens
cgtggtggaggctagctacaacgaggacgtctt

SEQ ID NO: 42 - DNA, 33 bp, Homo sapiens
gggggtagaggctagctacaacgaggagagggg

SEQ ID NO: 43 - DNA, 33 bp, Homo sapiens
ggaggaggaggctagctacaacgagaggccggg

SEQ ID NO: 44 - DNA, 33 bp, Homo sapiens
gccccccgaggctagctacaacgaaaggaggag

SEQ ID NO: 45 - DNA, 33 bp, Homo sapiens
ccggggagaggctagctacaacgagtccttcgg

SEQ ID NO: 46 - DNA, 33 bp, Homo sapiens
ggacagcgaggctagctacaacgagggtccggg

SEQ ID NO: 47 - DNA, 33 bp, Homo sapiens
tggggtggaggctagctacaacgaagcgatggg

SEQ ID NO: 48 - DNA, 33 bp, Homo sapiens
cttgaggcaggctagctacaacgatctttctcg

SEQ ID NO: 49 - DNA, 33 bp, Homo sapiens
cacctggtaggctagctacaacgattgaggcac

SEQ ID NO: 50 - DNA, 33 bp, Homo sapiens
gcaggggcaggctagctacaacgactggtactt

SEQ ID NO: 51 - DNA, 33 bp, Homo sapiens
ccagcttcaggctagctacaacgagctgtcggg

SEQ ID NO: 52 - DNA, 33 bp, Homo sapiens
gtgggacgaggctagctacaacgatccagcttc

SEQ ID NO: 53 - DNA, 33 bp, Homo sapiens
ggagtgggaggctagctacaacgagactccagc

SEQ ID NO: 54 - DNA, 33 bp, Homo sapiens
atgctgccaggctagctacaacgagggagtggg

SEQ ID NO: 55 - DNA, 33 bp, Homo sapiens
gggcggtcaggctagctacaacgagctgccacg

SEQ ID NO: 56 - DNA, 33 bp, Homo sapiens
gaggctccaggctagctacaacgaccagggcgg

SEQ ID NO: 57 - DNA, 33 bp, Homo sapiens
gtgggtcgaggctagctacaacgagaggaggct

SEQ ID NO: 58 - DNA, 33 bp, Homo sapiens
aggtggtgaggctagctacaacgaggggtggtg

SEQ ID NO: 59 - DNA, 33 bp, Homo sapiens
actcgggcaggctagctacaacgagtagggcgg

SEQ ID NO: 60 - DNA, 33 bp, Homo sapiens
ggagctgtaggctagctacaacgatcgggcacg

SEQ ID NO: 61 - DNA, 33 bp, Homo sapiens
ggacttgcaggctagctacaacgaccgaagccg

SEQ ID NO: 62 - DNA, 33 bp, Homo sapiens
gggcctggaggctagctacaacgattgcatccg

SEQ ID NO: 63 - DNA, 33 bp, Homo sapiens
tgtgctggaggctagctacaacgacgggccttg

SEQ ID NO: 64 - DNA, 33 bp, Homo sapiens
gttcacacaggctagctacaacgatccctgcct

SEQ ID NO: 65 - DNA, 33 bp, Homo sapiens
cagttcacaggctagctacaacgaactccctgc

SEQ ID NO: 66 - DNA, 33 bp, Homo sapiens
cacagttcaggctagctacaacgaacactccct

SEQ ID NO: 67 - DNA, 33 bp, Homo sapiens
gttgccccaggctagctacaacgaagttcacac

SEQ ID NO: 68 - DNA, 33 bp, Homo sapiens
tcgccgccaggctagctacaacgaagtggggtc

SEQ ID NO: 69 - DNA, 33 bp, Homo sapiens
cccgtgccaggctagctacaacgactcgccgcc

SEQ ID NO: 70 - DNA, 33 bp, Homo sapiens
ggcgttgcaggctagctacaacgaaggtagtgt

SEQ ID NO: 71 - DNA, 33 bp, Homo sapiens
tggcttctaggctagctacaacgagccctcgtc

SEQ ID NO: 72 - DNA, 33 bp, Homo sapiens
gggctctgaggctagctacaacgagcctggctt

SEQ ID NO: 73 - DNA, 33 bp, Homo sapiens
gggaccccaggctagctacaacgacggagcccg

SEQ ID NO: 74 - DNA, 33 bp, Homo sapiens
ggtgggggaggctagctacaacgacccaccgga

SEQ ID NO: 75 - DNA, 33 bp, Homo sapiens
ggcgggggaggctagctacaacgaccgagggcc

SEQ ID NO: 76 - DNA, 33 bp, Homo sapiens
gggctgggaggctagctacaacgagggcaggga

SEQ ID NO: 77 - DNA, 33 bp, Homo sapiens
cgtcgaggaggctagctacaacgaccgcccctc

SEQ ID NO: 78 - DNA, 33 bp, Homo sapiens
gggctggcaggctagctacaacgacttcccgta

SEQ ID NO: 79 - DNA, 33 bp, Homo sapiens
cgatgcccaggctagctacaacgaccggggcgg

SEQ ID NO: 80 - DNA, 33 bp, Homo sapiens
gctccacgaggctagctacaacgagcccatccg

SEQ ID NO: 81 - DNA, 33 bp, Homo sapiens
ccggctccaggctagctacaacgagatgcccat

SEQ ID NO: 82 - DNA, 33 bp, Homo sapiens
tctccgcaaggctagctacaacgaccggctcca

SEQ ID NO: 83 - DNA, 33 bp, Homo sapiens
ccgtcagcaggctagctacaacgagtctccgca

SEQ ID NO: 84 - DNA, 33 bp, Homo sapiens
tccccggcaggctagctacaacgacggctcggt

SEQ ID NO: 85 - DNA, 33 bp, Homo sapiens
cccccgcgaggctagctacaacgagctcgtccg

SEQ ID NO: 86 - DNA, 33 bp, Homo sapiens
gtagggagaggctagctacaacgacccaggctg

SEQ ID NO: 87 - DNA, 33 bp, Homo sapiens
gggcgggcaggctagctacaacgacaaggcgcc

SEQ ID NO: 88 - DNA, 33 bp, Homo sapiens
cgggaaggaggctagctacaacgatcgcccgcg

SEQ ID NO: 89 - DNA, 33 bp, Homo sapiens
tagtcctcaggctagctacaacgagcggccccg

SEQ ID NO: 90 - DNA, 33 bp, Homo sapiens
tccccgacaggctagctacaacgactccagtcc

SEQ ID NO: 91 - DNA, 33 bp, Homo sapiens
tttccccgaggctagctacaacgaacctccagt

SEQ ID NO: 92 - DNA, 33 bp, Homo sapiens
tgagcgcgaggctagctacaacgacctcagttt

SEQ ID NO: 93 - DNA, 33 bp, Homo sapiens
ggaccacaaggctagctacaacgaaggtggttg

SEQ ID NO: 94 - DNA, 33 bp, Homo sapiens
cttggaccaggctagctacaacgaaacaggtgg

SEQ ID NO: 95 - DNA, 33 bp, Homo sapiens
aaacttggaggctagctacaacgacacaacagg

SEQ ID NO: 96 - DNA, 33 bp, Homo sapiens
ctgattaaaggctagctacaacgattggaccac

SEQ ID NO: 97 - DNA, 33 bp, Homo sapiens
tggtgctgaggctagctacaacgataaacttgg

SEQ ID NO: 98 - DNA, 33 bp, Homo sapiens
tgatgatcaggctagctacaacgactctgtctg

SEQ ID NO: 99 - DNA, 33 bp, Homo sapiens
tggtgatgaggctagctacaacgacatctctgt

SEQ ID NO: 100 - DNA, 33 bp, Homo sapiens
gcttggtgaggctagctacaacgagatcatctc

SEQ ID NO: 101 - DNA, 33 bp, Homo sapiens
atgggaacaggctagctacaacgaccgccgtcc

SEQ ID NO: 102 - DNA, 33 bp, Homo sapiens
gaatgggaaggctagctacaacgaatccgccgt

SEQ ID NO: 103 - DNA, 33 bp, Homo sapiens
tgacaggaaggctagctacaacgagggaacatc

SEQ ID NO: 104 - DNA, 33 bp, Homo sapiens
agtaaatgaggctagctacaacgaaggaatggg

SEQ ID NO: 105 - DNA, 33 bp, Homo sapiens
cacagtaaaggctagctacaacgagacaggaat

SEQ ID NO: 106 - DNA, 33 bp, Homo sapiens
gcccggccaggctagctacaacgaagtaaatga

SEQ ID NO: 107 - DNA, 33 bp, Homo sapiens
ccacaaacaggctagctacaacgacctgtagtg

SEQ ID NO: 108 - DNA, 33 bp, Homo sapiens
gtccacaaaggctagctacaacgaatcctgtag

SEQ ID NO: 109 - DNA, 33 bp, Homo sapiens
ccacgtccaggctagctacaacgaaaacatcct

SEQ ID NO: 110 - DNA, 33 bp, Homo sapiens
ccaagaccaggctagctacaacgagtccacaaa

SEQ ID NO: 111 - DNA, 33 bp, Homo sapiens
ccaccaagaggctagctacaacgacacgtccac

SEQ ID NO: 112 - DNA, 33 bp, Homo sapiens
gctggtccaggctagctacaacgacaagaccac

SEQ ID NO: 113 - DNA, 33 bp, Homo sapiens
gctctggtaggctagctacaacgacgccagtgg

SEQ ID NO: 114 - DNA, 33 bp, Homo sapiens
ctgcacccaggctagctacaacgattgccgctc

SEQ ID NO: 115 - DNA, 33 bp, Homo sapiens
cacactgcaggctagctacaacgaccacttgcc

SEQ ID NO: 116 - DNA, 33 bp, Homo sapiens
ctttccacaggctagctacaacgatgcacccac

SEQ ID NO: 117 - DNA, 33 bp, Homo sapiens
gcctttccaggctagctacaacgaactgcaccc

SEQ ID NO: 118 - DNA, 33 bp, Homo sapiens
ttcctggcaggctagctacaacgagctgccctc

SEQ ID NO: 119 - DNA, 33 bp, Homo sapiens
gtggacgtaggctagctacaacgaaggcggttt

SEQ ID NO: 120 - DNA, 33 bp, Homo sapiens
ccgggtggaggctagctacaacgagtacaggcg

SEQ ID NO: 121 - DNA, 33 bp, Homo sapiens
cctggcgcaggctagctacaacgaccagtgcgc

SEQ ID NO: 122 - DNA, 33 bp, Homo sapiens
caaatgaaaggctagctacaacgattcctggcg

SEQ ID NO: 123 - DNA, 33 bp, Homo sapiens
tttcccaaaggctagctacaacgagaaacttcc

SEQ ID NO: 124 - DNA, 33 bp, Homo sapiens
attgttggaggctagctacaacgagcccccttg

SEQ ID NO: 125 - DNA, 33 bp, Homo sapiens
tgggtcacaggctagctacaacgatgttggacg

SEQ ID NO: 126 - DNA, 33 bp, Homo sapiens
tctgggtcaggctagctacaacgaattgttgga

SEQ ID NO: 127 - DNA, 33 bp, Homo sapiens
gcacaatcaggctagctacaacgactgggtcac

SEQ ID NO: 128 - DNA, 33 bp, Homo sapiens
ggagcacaaggctagctacaacgacatctgggt

SEQ ID NO: 129 - DNA, 33 bp, Homo sapiens
actggagcaggctagctacaacgaaatcatctg

SEQ ID NO: 130 - DNA, 33 bp, Homo sapiens
atggagggaggctagctacaacgatggagcaca

SEQ ID NO: 131 - DNA, 33 bp, Homo sapiens
tggtacttaggctagctacaacgaggagggact

SEQ ID NO: 132 - DNA, 33 bp, Homo sapiens
gggctggtaggctagctacaacgattatggagg

SEQ ID NO: 133 - DNA, 33 bp, Homo sapiens
tcaacgataggctagctacaacgagcagccggg

SEQ ID NO: 134 - DNA, 33 bp, Homo sapiens
cctcaacgaggctagctacaacgaatgcagccg

SEQ ID NO: 135 - DNA, 33 bp, Homo sapiens
tcacctcaaggctagctacaacgagatatgcag

SEQ ID NO: 136 - DNA, 33 bp, Homo sapiens
cgtcgttcaggctagctacaacgactcaacgat

SEQ ID NO: 137 - DNA, 33 bp, Homo sapiens
gtaaagataggctagctacaacgagcgtgttgg

SEQ ID NO: 138 - DNA, 33 bp, Homo sapiens
aagtaaagaggctagctacaacgaatgcgtgtt

SEQ ID NO: 139 - DNA, 33 bp, Homo sapiens
ggcaatgaaggctagctacaacgatgggtttct

SEQ ID NO: 140 - DNA, 33 bp, Homo sapiens
tcacggcaaggctagctacaacgagaactgggt

SEQ ID NO: 141 - DNA, 33 bp, Homo sapiens
aggcagtcaggctagctacaacgaggcaatgaa

SEQ ID NO: 142 - DNA, 33 bp, Homo sapiens
atctcggcaggctagctacaacgatctggtagg

SEQ ID NO: 143 - DNA, 33 bp, Homo sapiens
gctgagtaaggctagctacaacgactcggcatt

SEQ ID NO: 144 - DNA, 33 bp, Homo sapiens
tattatcaaggctagctacaacgatttcagctg

SEQ ID NO: 145 - DNA, 33 bp, Homo sapiens
gggttattaggctagctacaacgacaattttca

SEQ ID NO: 146 - DNA, 33 bp, Homo sapiens
aaggggttaggctagctacaacgatatcaattt

SEQ ID NO: 147 - DNA, 33 bp, Homo sapiens
ctcccggaaggctagctacaacgacctttggca

SEQ ID NO: 148 - DNA, 33 bp, Homo sapiens
gtacatggaggctagctacaacgatcaaagttc

(19) United States
(12) Patent Application Publication
(10) Pub. No.: US 2021/0299041 A1
(43) Pub. Date: Sep. 30, 2021

(54) Aerosol-generating device for the inhalation administration of an antisense-molecule-containing composition

(71) Applicant: Sterna Biologicals GmbH & Co. KG, Marburg (DE)

(72) Inventor: Jonas Renz, Munich (DE)

(73) Assignee: Sterna Biologicals GmbH & Co. KG, Marburg (DE)

(21) Appl. No.: 17/263,910

(22) PCT Filed: Jul. 29, 2019
(86) PCT No.: PCT/EP2019/070399

§ 371 Date: Jan. 28, 2021

(30) Foreign Application Priority Data
Jul. 30, 2018 (EP) 18186429.9

(51) Int. Cl. A61K 9/00; A61K 31/7088; C12N 15/113; A61M 11/00

(52) U.S. Cl. CPC A61K 9/0078; A61K 31/7088; C12N 2310/11; A61M 11/005; C12N 15/113

(57) ABSTRACT

The invention relates to the use of an aerosol-generating device for generating an aerosol of a composition, for administering the composition by inhalation, wherein the composition comprises at least one antisense-molecule. The aerosol-generating device has a membrane having a perforation, wherein the composition for generating the aerosol is brought into contact with the membrane, and the membrane of the aerosol-generating device are caused to vibrate. The perforation is formed of at least one group of passage openings, wherein each passage opening of a group has a diameter of at least 3 μm and at most 5 μm at the narrowest point of the passage opening, and wherein the at least one group of passage openings comprises at maximum 500 passage openings per mm 2 of the membrane.

The invention relates to the use of an aerosolizer for generating an aerosol of an antisense molecule-containing composition, for administering the composition by inhalation. The invention relates more particularly to the use as claimed in claim 1.

Unless otherwise indicated, the terms “expression” and “gene expression” are used synonymously hereinafter. Furthermore, the term “aerosol” refers hereinafter to a mixture of solid or liquid particles in gases or air, whereas the term “mist” refers to finely distributed drops of liquid in the air.

Inflammations of the respiratory tract are the most frequently occurring respiratory tract diseases. They often lead to chronic inflammations. One example of such inflammations are allergies, which are brought about by a severely disproportionate immune response to antigens that are normally harmless to humans, such as pollen, animal hairs, foods, mites, preservatives, dyes, cleaning products etc. Other typical respiratory tract diseases which lead to chronic inflammations are, for example, autoimmune diseases such as asthma or chronic obstructive pulmonary diseases (COPD). The progression of the initial inflammatory reaction through to the chronic inflammation involves the participation of processes of destruction and of modification. A consequence is the loss of tolerance—that is, of the capacity not to attack endogenous cells, commensal causatives or insignificant allergens.

Components of both the innate and acquired immune system are involved in the immune response. An important role is played in this context by, in particular, the Th1 cell-specific transcription factor Tbet, which regulates the specific development of Th1 cells, and the Th2 cell-specific transcription factor GATA-3, which regulates the specific development of Th2 cells.

In the healthy body there is an equilibrium between Th1 and Th2 cells. This equilibrium may be disturbed by certain influences. In the case of a chronic inflammation, for example, the equilibrium between Th1 and Th2 cells is shifted in favor of the Th2 cells by the expression of GATA-3 and/or by the inhibition of Tbet. The Th2 cells become predominant, this in turn being typical of many chronic inflammatory diseases in the late phase.

In other cases, the Th1 cells are predominant, when there is inhibition of GATA-3 or when the expression of Tbet produces a rising Th1 cell level.

One therapeutic method, then, involves specifically inhibiting or “switching off” the transcription factors GATA-3 and/or Tbet when there is predominance of Th1 or Th2 cells, respectively. It is possible in this way to restore the equilibrium between the two types of cell. One possibility here is that of targeted inhibition of the gene expression of the transcription factors GATA-3 or Tbet, respectively, through the use, for instance, of suitable antisense molecules.

WO 2005/033314 A2 describes the mode of action of various GATA-3-specific or Tbet-specific antisense molecules in the form of DNAzymes. The disclosure content of WO 2005/033314 is therefore regarded as technological background to the present invention.

In the case of respiratory tract diseases in particular, the respective active ingredients are administered by inhalation, with “inhalation” referring to the breathing-in of aerosols or gaseous active ingredients. Inhalation is an established method enabling direct access to various regions of the lungs. Intrapulmonary and transpulmonary medicament administration of the active ingredients is possible in this way. The option of taking a drug by way of inhalation, by the lungs or by the targeted cells in the lungs, may represent an important factor in the efficacy of a drug. Among the factors to play a part in this context is the size of the particles to be inhaled. This size is characterized by way of the MMD (“mass median diameter”) or by way of the MMAD (“mass median aerodynamic diameter”).

There are numerous known inhalation devices, with three types of device being commonly used, and each differing in their operation: metered dose inhalers, dry powder inhalers, and, in particular, aerosolizers. Fundamentally it is the case that tabletop devices are a disadvantage, since when used there is no targeted nebulization into the lungs. The number of droplets the patient breathes in is dependent instead on his or her breathing actions.

Aerosolizers, also referred to as nebulizers, separate fine droplets (also referred to below as drops or particles) from a liquid reservoir or a solution. This forms an aerosol, which can be inhaled by the patient. There are three functional principals by which the nebulizers are classified. With jet nebulizers, a strong flow of air generates underpressure at a nozzle and so draws droplets from a capillary system. Because the droplets have different sizes, a baffle plate is used in order to retain droplets that are too large.

With ultrasonic nebulizers, the droplets are generated by ultrasound. The higher the frequency of the ultrasound, the finer the droplets.

Membrane nebulizers feature a very thin membrane having a multiplicity of microholes or passages—one membrane may have well above one thousand such passages. The membrane is caused to vibrate, and each passage thereby acts as a pump and produces droplets having a defined MMAD.

Antisense molecules are understood generally to be molecules which either are composed of usually single-stranded RNA and/or DNA oligonucleotides or have at least one such oligonucleotide constituent. Oligonucleotides in turn are molecules composed of a few nucleotides, such as, for example, primers which are used in the polymerase chain reaction (PCR). Antisense molecules usually possess oligonucleotides whose base sequence is complementary to a cellular, viral or synthetic RNA or DNA molecules, and are able to bind to these molecules via Watson-Crick base pairing. This characteristic can be utilized, for example, in order to inhibit the function of an arbitrary target mRNA, through the specific binding of the antisense molecules to the target mRNA, for instance. Both in research and in therapeutic application, antisense molecules are used in order to achieve targeted attenuation or complete suppression of the expression of a gene. Also known for a number of years have been antisense molecules which are not constructed on the basis of nucleic acids. Such molecules then as a general rule comprise nucleic acid analogs which match with true nucleic acids in terms of their capacity for base pairing.

For antisense molecule-containing compositions, at the present time, there are no known suitable governing conditions or parameters for the use of such a composition via an aerosolizer for the inhalation treatment of a patient suffering from a respiratory tract disease associated with chronic inflammation. Instead, in the use of an antisense molecule-containing composition, it is possible to observe the phenomenon that the membranes of the particular aerosolizers used may suffer blocking after a short time, since antisense molecule-containing compositions or solutions have a high viscosity.

It is the object of the invention, therefore, to enable the use of an aerosolizer with which an antisense molecule-containing composition can be administered by inhalation for the purpose of treating a patient suffering from a respiratory tract disease associated with chronic inflammation. The intention more particularly is that the antisense molecule-containing composition should be able by means of the aerosolizer to be administered simply, reliably, and reproducibly and also be atomized in a defined way.

This object is achieved in accordance with the invention by the use of an aerosolizer for generating an aerosol, and of a correspondingly adapted composition for administration by inhalation, where the composition comprises at least one antisense molecule, where the composition, for generating the aerosol, is contacted with the membrane, and the membrane of the aerosolizer is set in vibration, where the perforation is formed by at least one group of passages, where each passage of a group has a diameter at its narrowest point of at least 3 μm and at most 5 μm and where the at least one group of passages comprises at maximum 500 passages per mm² of the membrane. The antisense molecule-containing composition is more particularly a liquid or a solution. With preference each passage of the membrane at its narrowest point has a diameter of at least 3 μm and at most 5 μm. The passages or microholes here may have differing geometries. They may for example be slitted, angular or oval or may have a hole geometry. The microholes used in the membrane are presently, in particular, holes, there being at least 100 microholes or passages per membrane. Further, the membrane is excited by a piezoelectric element and set in oscillations. The antisense molecule is selected from the group encompassing DNAzymes, siRNAs, asDNAs or Ribozymes. In the context of the present specification, preference is given to using DNAzymes as antisense molecules. Alternatively, however, the antisense molecule may also from the group of molecules encompassing phosphorothioate oligonucleotides (PTO), 2′-fluoro-RNAs, FANAs, 2′-O-methyloligonucleotides, 2′-O-methoxyethyloligonucleotides, constrained ethyl oligonucleotides (cEt), locked nucleic acids (LNA), 2′-0,4′-C-ethylene-bridged nucleic acid (ENA), peptide nucleic acids (PNA), morpholinos, Ugimers, tricyclo-DNA, DNA primers, or aptamers.

A key advantage arising from the use of the aerosolizer of the invention is that the antisense molecule-containing composition can be converted into an aerosol whose particles have a VMD MV (volume median diameter) of around 4 μm. Because of impactation forces by larger particles, only an aerosol with particles smaller than 5 μm is able to reach chronic inflammation in the middle and lower respiratory tracts, so that corresponding antisense molecules, especially DNAzymes, reach the site of action and take therapeutic effect there. Accordingly, an effective, simple and reliable way is provided of administering the viscous antisense molecule-containing composition through inhalation.

With the use in the invention of an aerosolizer having a vibrating membrane with microholes each having a diameter of 3 μm to 5 μm, it is possible for antisense molecule-containing compositions, which in general have a high viscosity, to be converted reliably into a therapeutically effective and inhalable aerosol. The use according to the invention enables the patient, moreover, to receive always a defined dose of active ingredient per activation event of the aerosolizer or per inhalation phase. An antisense molecule-containing composition can therefore be administered safely, reliably and reproducibly.

The feature whereby the group of passages comprises at maximum 500 passages per mm² of the membrane supports the advantageous effect on droplet formation of the passages having a diameter of at least 3 μm and at most 5 μm, namely that the antisense molecule-containing composition can be converted into an aerosol whose particles have a VMD MV of around 4 μm. Hence membranes having a hole density of more than 500 passages per mm² may result in the droplets or particles formed fusing, aggregating or combining to form larger particles immediately after emergence from the passages of the membrane. This would have the drawback in turn that the particles or drops, because of impactation forces, would not reach their corresponding site of action, or at least would do so to a reduced extent. The antisense molecules and in particular the DNAzymes would therefore be unable to develop their therapeutic effect at the site at which the chronic inflammation is present. Conversely, the use in the invention of aerosolizers having membranes which comprise at maximum 500 passages per mm² provides an effective, simple and safe way of administering the viscous, antisense molecule-containing composition through inhalation. The atomization of the composition to form an aerosol with droplets of the preferred size is boosted, furthermore, by the vibration of the membrane.

This aerosolizer is preferably a portable and convenient device which can be deployed even in emergency situations. Energy is supplied to the apparatus via cable, via one or more batteries, or inductively. As a result of the use according to the invention, moreover, the generated aerosol of the antisense molecule-containing composition is free from harmful gaseous propellants.

The recited use reduces the risks of side effects in the rest of the body; moreover, the locally targeted activity of the antisense molecules necessitates a lower dose of active ingredient per administration than is the case with non-specific administration forms.

Provision may be made, moreover, for the aerosolizer to have a chip technology such as Bluetooth®, for example, in order for instance to monitor patient observance and compliance.

According to one advantageous development, moreover, provision may be made for the membrane to be coupled to a piezoelectric crystal or transducer which sets the membrane in vibration. This embodiment allows the membrane to be vibrated, with a vibration frequency preferably of 10 kHz to 150 kHz. The vibration causes the membrane to move up and down a few micrometers and so to act like a “micropump”, by pressing the antisense molecule-containing composition as an aerosol in the direction of the exit from the aerosolizer.

Provision may be made in the invention for the passages to be conical, in which case the diameter of each conical passage decreases in the direction of the region of the passage at which the aerosol emerges from the passage. This embodiment ensures a maximally and uniform size of the droplets in the aerosol.

It is also an advantage for the perforation in the membrane to have at least two groups of passages.

In a particularly preferred variant in the context of the use of an aerosolizer, the membrane has a diameter of 1 mm to 8 mm, particular preference being given to a membrane having a diameter of 6 mm to 8 mm. In a further advantageous embodiment, the membrane is produced from a material which comprises silicon and/or at least one metal, the metal more particularly being an element from the group of stainless steel, nickel, palladium, cobalt, rust-free steel and/or being an alloy of at least two of the elements. A membrane produced from rust-free steel or from nickel is particularly preferred. Furthermore, the perforated membrane of the aerosolizer may be circular, oval or angular. The use of a membrane made from such a material gives the membrane a high stability. At the same time, such a membrane is favorable to produce. An aerosolizer featuring such a membrane, moreover, is robust and therefore long-lived.

It is an advantage, moreover, if the composition has an antisense molecule concentration of below 75 mg/ml. It can accordingly be used ideally for administration by inhalation in the treatment of patients suffering from a respiratory tract disease associated with chronic inflammation. This is so particularly because compositions with antisense molecule concentrations exceeding 75 mg/ml have such a high viscosity that they are difficult or even impossible to administer as an aerosol using an aerosolizer, because the passages in the perforation of the membrane are easily clogged because of the high viscosity of the composition. Compositions having an antisense molecule concentration of above 75 mg/ml, moreover, exhibit a loss of active ingredient of around 6%. This is because of the three-dimensional structure of the antisense molecules, which readily associate with one another and so form a polymer structure. This property is observed to particular effect with DNAzymes.

It is therefore of particular advantage if the composition has an antisense molecule concentration of 20 mg/ml to 50 mg/ml. Compositions having an antisense molecule concentration of 20 mg/ml to 50 mg/ml display an optimum activity effect in the context of the use according to the invention with an aerosolizer.

Provision may also be made for the composition to have a viscosity which is lower than 3.5 mPa·s. In the case of a composition having a viscosity of above 3.5 mPa·s, an aerosolizer is not readily able to generate the pressure needed for the atomization or misting of the composition. As a result, in turn, the passages or holes in the membrane become clogged or fouled. This risk is significantly minimized by a composition whose antisense molecule concentration is lower than 75 mg/ml. A composition with a viscosity of this kind can be administered by inhalation via the use according to the invention without problems and without significance loss of active ingredient.

In a preferred variant connected with the use of an aerosolizer in the invention, the group of passages comprises at maximum 200 passages per mm² of the membrane. In a particularly preferred variant, the group of passages comprises at maximum 50 passages per mm² of the membrane. The limitation on hole density to 200 or even to 50 passages per mm² reinforces the advantageous effect of the hole density whereby the fusion, aggregation or combination of the droplets following their emergence from the passages is prevented. The use in the invention of aerosolizers having membranes which comprise at maximum 200 or at maximum 50 passages per mm² therefore enables an effective, simple and safe way of administering the viscous, antisense molecule-containing composition by inhalation.

According to one advantageous embodiment of the use, the composition comprises at least one antisense molecule which is selected from the group encompassing DNAzymes, siRNAs, asDNAs or Ribozymes. Provision may be made more particularly for the at least one antisense molecule to specifically downregulate the expression of GATA-3 or specifically downregulate the expression of Tbet. The antisense molecules are preferably DNAzymes. The specific antisense molecules here bind and cleave the mRNAs of the transcription factors GATA-3 and/or Tbet in vivo, the proteins of which are in turn central key molecules for the development of Th1- and, respectively, Th2-dependent chronic inflammatory diseases. As a result of this cleaving of the mRNAs, the mRNAs can no longer be translated to functional proteins. Accordingly, the level of GATA-3 or Tbet protein is significantly minimized.

According to one preferred variant embodiment, the at least one antisense molecule is a DNAzyme. The DNAzyme is more preferably selected from a group encompassing the DNAzymes hdg1 to hdg70, or from a group encompassing the DNAzymes td1 to td78. In particular, the DNAzyme has the hdg40 sequence (GTGGATGGAggctagctacaacgaGTCTTGGAG). Such a sequence exhibits particularly high enzyme activity and cleaves GATA-3 mRNA with a high specificity and also high efficiency. Accordingly, a DNAzyme with the hgd40 sequence (SEQ ID No. 40) is outstandingly suitable for the targeted and effective treatment of respiratory tract diseases which are Th2-dependent, and which therefore correspond with an elevated GATA-3 level. According to one advantageous development, the composition may comprise at least one nuclease inhibitor, in which case the at least one nuclease inhibitor more particularly specifically deactivates deoxyribonucleases and is thus a DNase inhibitor. A nuclease inhibitor of this kind protects the at least one DNAzyme from enzymatic breakdown by DNases.

One example of an antisense molecule-containing composition is represented in the following table:

| Substance | [% w/w] |
|---|---|
| Antisense molecule (DNAzyme) | 0.01-0.75 |
| Nuclease inhibitor (e.g., DNase inhibitor) | variable |

It is possible, additionally, for the composition to comprise at least one salt and/or at least one cation. A composition of this kind is advantageously suitable for application in the therapy of patients suffering from a respiratory tract disease associated with chronic inflammation, since the composition consequently has a physiologically favorable medium and is therefore readily tolerated by patients. Provided more particularly in this context is a phosphate-buffered saline (PBS) solution.

Alternatively, however, other buffered solutions are also used that contain nucleic acids in solution in a physiologically favorable medium, such as, for example, TE buffer. Provided accordingly is an organic and/or inorganic addition, the addition being preferably selected from the group encompassing the compounds sodium chloride (NaCl), potassium chloride (KCl), disodium hydrogen phosphate (Na₂HPO4), disodium hydrogen phosphate dihydrate, potassium dihydrogen phosphate (KH₂PO4), TRIS and EDTA. The cation in this case may be selected from the group encompassing Na, Mg, K, Li, Ca, Fe, Cu and Ag. Provided alternatively as well is an organic cation, such as Mg(N(SO₂CF₃)₂)₂ or Mg(OSO₂CF₃)₂, for example. Alternatively, a divalent cation is used. A composition with this kind of buffering is particularly suitable for protecting the antisense molecules, since the organic and/or inorganic addition stabilizes the antisense molecule and protects it from enzymatic breakdown. Furthermore, the salt allows the composition to be taken up well into the target cells. The divalent cations may act as co-factors of the antisense molecules to boost the antisense molecule activity, because the antisense molecules have a catalytic domain which is dependent on divalent cations (preferably Mg²⁺). Divalent cations therefore act as “enhancers” or boosters. According to one advantageous development, the composition may comprise at least one organic and/or inorganic addition and/or a solubilizer and/or a preservative. The solubilizer in this case is used in particular for complexation. Furthermore, it improves the solvent properties of the composition. Solubilizers provided may preferably be glycerol derivatives and/or polyethylene glycols or else lecithins. The preservative may be paraben, for example.

Possible additional components of the antisense molecule-containing composition are as follows:

| Substance | [% w/w] |
|---|---|
| Salt and/or a cation | variable |
| (e.g., Na, Mg, K, Li, Ca, Fe, Cu, Ag, HPO₄^{2+,} H₂PO⁴⁻) | |
| EDTA | variable |
| TRIS | variable |
| Solubilizer | variable |
| (e.g., glycerol derivatives, polyethylene glycols, lecithins) | |
| Preservative | variable |
| (e.g., paraben) | |

Further provided may be additions whose effect, for example, is that of changing the surface tension of the composition or of lowering the viscosity of the composition.

The effects and advantages set out above arise in particular in the context of the use of an aerosolizer for generating an aerosol of a composition for administration by inhalation in the treatment of a patient who is suffering from a respiratory tract disease associated with chronic inflammation.

Further features, details and advantages of the invention will emerge from the wording of the claims and also from the description hereinafter of exemplary embodiments and figures.

### EXEMPLARY EMBODIMENTS

Table 1 below shows the GATA-3 specific DNAzymes hgd1-70 with the sequence IDs as per the sequence protocol:

| Name | Sequence |
|---|---|
| hgd1 | 5′-TCGGTCAGAggctagctacaacgaTGCGTTGCT-3′ |
| hgd2 | 5′-GGCGTACGAggctagctacaacgaCTGCTCGGT-3′ |
| hgd3 | 5′-GGCGGCGTAggctagctacaacgaGACCTGCTC-3′ |
| hgd4 | 5′-CTCGGGTCAggctagctacaacgaCTGGGTAGC-3′ |
| hgd5 | 5′-TCCTCTGCAggctagctacaacgaCGGGGTCCT-3′ |
| hgd6 | 5′-ACTCTGCAAggctagctacaacgaTCTGCGAGC-3′ |
| hgd7 | 5′-GGGCGACGAggctagctacaacgaTCTGCAATT-3′ |
| hgd8 | 5′-AAGGGGCGAggctagctacaacgaGACTCTGCA-3′ |
| hgd9 | 5′-AAAACGGGAggctagctacaacgaCAGGTTGTA-3′ |
| hgd10 | 5′-AGAATAAAAggctagctacaacgaGGGACCAGG-3′ |
| hgd11 | 5′-ATGGCAGAAggctagctacaacgaAAAACGGGA-3′ |
| hgd12 | 5′-AACTGGGTAggctagctacaacgaGGCAGAATA-3′ |
| hgd13 | 5′-ATCCAAAAAggctagctacaacgaTGGGTATGG-3′ |
| hgd14 | 5′-AGGGGAAGAggctagctacaacgaAAAAATCCA-3′ |
| hgd15 | 5′-TTTTAAAAAggctagctacaacgaTATCTTGGA-3′ |
| hgd16 | 5′-GTGGGGGGAggctagctacaacgaGGGAAGGCT-3′ |
| hgd17 | 5′-GTTGAATGAggctagctacaacgaTTGCTTTCG-3′ |
| hgd18 | 5′-GTCGTTGAAggctagctacaacgaGATTTGCTT-3′ |
| hgd19 | 5′-GGCCCGGAAggctagctacaacgaCCGCGCGCG-3′ |
| hgd20 | 5′-TCACCTCCAggctagctacaacgaGGCCTCGGC-3′ |
| hgd21 | 5′-CCGCCGTCAggctagctacaacgaCTCCATGGC-3′ |
| hgd22 | 5′-GGTGGCTCAggctagctacaacgaCCAGCGCGG-3′ |
| hgd23 | 5′-CGTTGAGCAggctagctacaacgaGGCGGGGTG-3′ |
| hgd24 | 5′-CCGCGTCCAggctagctacaacgaGTAGGAGTG-3′ |
| hgd25 | 5′-CAGCGGGTAggctagctacaacgaTGCGCCGCG-3′ |
| hgd26 | 5′-GCACATCCAggctagctacaacgaCTCCTCCGG-3′ |
| hgd27 | 5′-AAAAGCACAggctagctacaacgaCCACCTCCT-3′ |
| hgd28 | 5′-TAAAAAGCAggctagctacaacgaATCCACCTC-3′ |
| hgd29 | 5′-GACCGTCGAggctagctacaacgaGTTAAAAAG-3′ |
| hgd30 | 5′-TTGCCTTGAggctagctacaacgaCGTCGATGT-3′ |
| hgd31 | 5′-AGGGCGGGAggctagctacaacgaGTGGTTGCC-3′ |
| hgd32 | 5′-TGGCCCTGAggctagctacaacgaCGAGTTTCC-3′ |
| hgd33 | 5′-ACCTCTGCAggctagctacaacgaCGTGGCCCT-3′ |
| hgd34 | 5′-CGGAGGGTAggctagctacaacgaCTCTGCACC-3′ |
| hgd35 | 5′-GGCGGCACAggctagctacaacgaCTGGCTCCC-3′ |
| hgd36 | 5′-CGGGCGGCAggctagctacaacgaACCTGGCTC-3′ |
| hgd37 | 5′-AGGGATCCAggctagctacaacgaGAAGCAGAG-3′ |
| hgd38 | 5′-GGGTAGGGAggctagctacaacgaCCATGAAGC-3′ |
| hgd39 | 5′-GGGCTGAGAggctagctacaacgaTCCAGGGGG-3′ |
| hgd40 | 5′-GTGGATGGAggctagctacaacgaGTCTTGGAG-3′ |
| hgd41 | 5′-CGTGGTGGAggctagctacaacgaGGACGTCTT-3′ |
| hgd42 | 5′-GGGGGTAGAggctagctacaacgaGGAGAGGGG-3′ |
| hgd43 | 5′-GGAGGAGGAggctagctacaacgaGAGGCCGGG-3′ |
| hgd44 | 5′-GCCCCCCGAggctagctacaacgaAAGGAGGAG-3′ |
| hgd45 | 5′-CCGGGGAGAggctagctacaacgaGTCCTTCGG-3′ |
| hgd46 | 5′-GGACAGCGAggctagctacaacgaGGGTCCGGG-3′ |
| hgd47 | 5′-TGGGGTGGAggctagctacaacgaAGCGATGGG-3′ |
| hgd48 | 5′-CTTGAGGCAggctagctacaacgaTCTTTCTCG-3′ |
| hgd49 | 5′-CACCTGGTAggctagctacaacgaTTGAGGCAC-3′ |
| hgd50 | 5′-GCAGGGGCAggctagctacaacgaCTGGTACTT-3′ |
| hgd51 | 5′-CCAGCTTCAggctagctacaacgaGCTGTCGGG-3′ |
| hgd52 | 5′-GTGGGACGAggctagctacaacgaTCCAGCTTC-3′ |
| hgd53 | 5′-GGAGTGGGAggctagctacaacgaGACTCCAGC-3′ |
| hgd54 | 5′-ATGCTGCCAggctagctacaacgaGGGAGTGGG-3′ |
| hgd55 | 5′-GGGCGGTCAggctagctacaacgaGCTGCCACG-3′ |
| hgd56 | 5′-GAGGCTCCAggctagctacaacgaCCAGGGCGG-3′ |
| hgd57 | 5′-GTGGGTCGAggctagctacaacgaGAGGAGGCT-3′ |
| hgd58 | 5′-AGGTGGTGAggctagctacaacgaGGGGTGGTG-3′ |
| hgd59 | 5′-ACTCGGGCAggctagctacaacgaGTAGGGCGG-3′ |
| hgd60 | 5′-GGAGCTGTAggctagctacaacgaTCGGGCACG-3′ |
| hgd61 | 5′-GGACTTGCAggctagctacaacgaCCGAAGCCG-3′ |
| hgd62 | 5′-GGGCCTGGAggctagctacaacgaTTGCATCCG-3′ |
| hgd63 | 5′-TGTGCTGGAggctagctacaacgaCGGGCCTTG-3′ |
| hgd64 | 5′-GTTCACACAggctagctacaacgaTCCCTGCCT-3′ |
| hgd65 | 5′-CAGTTCACAggctagctacaacgaACTCCCTGC-3′ |
| hgd66 | 5′-CACAGTTCAggctagctacaacgaACACTCCCT-3′ |
| hgd67 | 5′-GTTGCCCCAggctagctacaacgaAGTTCACAC-3′ |
| hgd68 | 5′-TCGCCGCCAggctagctacaacgaAGTGGGGTC-3′ |
| hgd69 | 5′-CCCGTGCCAggctagctacaacgaCTCGCCGCC-3′ |
| hgd70 | 5′-GGCGTTGCAggctagctacaacgaAGGTAGTGT-3′ |

Table 2 below shows the Tbet-specific DNAzymes td1-78 with their sequence IDs as per the sequence protocol:

| Name | Sequence |
|---|---|
| td1 | 5′-TGGCTTCTAggctagctacaacgaGCCCTCGTC-3′ |
| td2 | 5′-GGGCTCTGAggctagctacaacgaGCCTGGCTT-3′ |
| td3 | 5′-GGGACCCCAggctagctacaacgaCGGAGCCCG-3′ |
| td4 | 5′-GGTGGGGGAggctagctacaacgaCCCACCGGA-3′ |
| td5 | 5′-GGCGGGGGAggctagctacaacgaCCGAGGGCC-3′ |
| td6 | 5′-GGGCTGGGAggctagctacaacgaGGGCAGGGA-3′ |
| td7 | 5′-CGTCGAGGAggctagctacaacgaCCGCCCCTC-3′ |
| td8 | 5′-GGGCTGGCAggctagctacaacgaCTTCCCGTA-3′ |
| td9 | 5′-CGATGCCCAggctagctacaacgaCCGGGGCGG-3′ |
| td10 | 5′-GCTCCACGAggctagctacaacgaGCCCATCCG-3′ |
| td11 | 5′-CCGGCTCCAggctagctacaacgaGATGCCCAT-3′ |
| td12 | 5′-TCTCCGCAAggctagctacaacgaCCGGCTCCA-3′ |
| td13 | 5′-CCGTCAGCAggctagctacaacgaGTCTCCGCA-3′ |
| td14 | 5′-TCCCCGGCAggctagctacaacgaCGGCTCGGT-3′ |
| td15 | 5′-CCCCCGCGAggctagctacaacgaGCTCGTCCG-3′ |
| td16 | 5′-GTAGGGAGAggctagctacaacgaCCCAGGCTG-3′ |
| td17 | 5′-GGGCGGGCAggctagctacaacgaCAAGGCGCC-3′ |
| td18 | 5′-CGGGAAGGAggctagctacaacgaTCGCCCGCG-3′ |
| td19 | 5′-TAGTCCTCAggctagctacaacgaGCGGCCCCG-3′ |
| td20 | 5′-TCCCCGACAggctagctacaacgaCTCCAGTCC-3′ |
| td21 | 5′-TTTCCCCGAggctagctacaacgaACCTCCAGT-3′ |
| td22 | 5′-TGAGCGCGAggctagctacaacgaCCTCAGTTT-3′ |
| td23 | 5′-GGACCACAAggctagctacaacgaAGGTGGTTG-3′ |
| td24 | 5′-CTTGGACCAggctagctacaacgaAACAGGTGG-3′ |
| td25 | 5′-AAACTTGGAggctagctacaacgaCACAACAGG-3′ |
| td26 | 5′-CTGATTAAAggctagctacaacgaTTGGACCAC-3′ |
| td27 | 5′-TGGTGCTGAggctagctacaacgaTAAACTTGG-3′ |
| td28 | 5′-TGATGATCAggctagctacaacgaCTCTGTCTG-3′ |
| td29 | 5′-TGGTGATGAggctagctacaacgaCATCTCTGT-3′ |
| td30 | 5′-GCTTGGTGAggctagctacaacgaGATCATCTC-3′ |
| td31 | 5′-ATGGGAACAggctagctacaacgaCCGCCGTCC-3′ |
| td32 | 5′-GAATGGGAAggctagctacaacgaATCCGCCGT-3′ |
| td33 | 5′-TGACAGGAAggctagctacaacgaGGGAACATC-3′ |
| td34 | 5′-AGTAAATGAggctagctacaacgaAGGAATGGG-3′ |
| td35 | 5′-CACAGTAAAggctagctacaacgaGACAGGAAT-3′ |
| td36 | 5′-GCCCGGCCAggctagctacaacgaAGTAAATGA-3′ |
| td37 | 5′-CCACAAACAggctagctacaacgaCCTGTAGTG-3′ |
| td38 | 5′-GTCCACAAAggctagctacaacgaATCCTGTAG-3′ |
| td39 | 5′-CCACGTCCAggctagctacaacgaAAACATCCT-3′ |
| td40 | 5′-CCAAGACCAggctagctacaacgaGTCCACAAA-3′ |
| td41 | 5′-CCACCAAGAggctagctacaacgaCACGTCCAC-3′ |
| td42 | 5′-GCTGGTCCAggctagctacaacgaCAAGACCAC-3′ |
| td43 | 5′-GCTCTGGTAggctagctacaacgaCGCCAGTGG-3′ |
| td44 | 5′-CTGCACCCAggctagctacaacgaTTGCCGCTC-3′ |
| td45 | 5′-CACACTGCAggctagctacaacgaCCACTTGCC-3′ |
| td46 | 5′-CTTTCCACAggctagctacaacgaTGCACCCAC-3′ |
| td47 | 5′-GCCTTTCCAggctagctacaacgaACTGCACCC-3′ |
| td48 | 5′-TTCCTGGCAggctagctacaacgaGCTGCCCTC-3′ |
| td49 | 5′-GTGGACGTAggctagctacaacgaAGGCGGTTT-3′ |
| td50 | 5′-CCGGGTGGAggctagctacaacgaGTACAGGCG-3′ |
| td51 | 5′-CCTGGCGCAggctagctacaacgaCCAGTGCGC-3′ |
| td52 | 5′-CAAATGAAAggctagctacaacgaTTCCTGGCG-3′ |
| td53 | 5′-TTTCCCAAAggctagctacaacgaGAAACTTCC-3′ |
| td54 | 5′-ATTGTTGGAggctagctacaacgaGCCCCCTTG-3′ |
| td55 | 5′-TGGGTCACAggctagctacaacgaTGTTGGACG-3′ |
| td56 | 5′-TCTGGGTCAggctagctacaacgaATTGTTGGA-3′ |
| td57 | 5′-GCACAATCAggctagctacaacgaCTGGGTCAC-3′ |
| td58 | 5′-GGAGCACAAggctagctacaacgaCATCTGGGT-3′ |
| td59 | 5′-ACTGGAGCAggctagctacaacgaAATCATCTG-3′ |
| td60 | 5′-ATGGAGGGAggctagctacaacgaTGGAGCACA-3′ |
| td61 | 5′-TGGTACTTAggctagctacaacgaGGAGGGACT-3′ |
| td62 | 5′-GGGCTGGTAggctagctacaacgaTTATGGAGG-3′ |
| td63 | 5′-TCAACGATAggctagctacaacgaGCAGCCGGG-3′ |
| td64 | 5′-CCTCAACGAggctagctacaacgaATGCAGCCG-3′ |
| td65 | 5′-TCACCTCAAggctagctacaacgaGATATGCAG-3′ |
| td66 | 5′-CGTCGTTCAggctagctacaacgaCTCAACGAT-3′ |
| td67 | 5′-GTAAAGATAggctagctacaacgaGCGTGTTGG-3′ |
| td68 | 5′-AAGTAAAGAggctagctacaacgaATGCGTGTT-3′ |
| td69 | 5′-GGCAATGAAggctagctacaacgaTGGGTTTCT-3′ |
| td70 | 5′-TCACGGCAAggctagctacaacgaGAACTGGGT-3′ |
| td71 | 5′-AGGCAGTCAggctagctacaacgaGGCAATGAA-3′ |
| td72 | 5′-ATCTCGGCAggctagctacaacgaTCTGGTAGG-3′ |
| td73 | 5′-GCTGAGTAAggctagctacaacgaCTCGGCATT-3′ |
| td74 | 5′-TATTATCAAggctagctacaacgaTTTCAGCTG-3′ |
| td75 | 5′-GGGTTATTAggctagctacaacgaCAATTTTCA-3′ |
| td76 | 5′-AAGGGGTTAggctagctacaacgaTATCAATTT-3′ |
| td77 | 5′-CTCCCGGAAggctagctacaacgaCCTTTGGCA-3′ |
| td78 | 5′-GTACATGGAggctagctacaacgaTCAAAGTTC-3′ |

The aerosolizers presently tested each used a composition having an antisense molecule concentration of below 75 mg/ml. Subsequently the size of the drops or droplets of the resultant aerosol was ascertained.

In a first test phase, aerosolizers with a perforated membrane were tested without a piezoelectric transducer. With these aerosolizers it was not possible to mist an antisense molecule-containing composition for treating a patient suffering from a respiratory tract disease associated with chronic inflammation.

In this case it was surprisingly discovered that an aerosol with droplets having a VMD MV of around 4 μm is formed through the use of a composition having an antisense molecule concentration of less than 75 mg/ml, together with an aerosolizer having vibrating membranes, the passages in the membranes having a diameter of at least 3 μm to at most 5 μm and the membranes having a diameter of 6 mm to 8 mm. The passages in the membranes do not become clogged. This phenomenon can be observed in particular for an antisense molecule concentration of between 20 mg/ml and 50 mg/ml.

Aerosolizers were tested with different materials of the perforated membrane: rust-free steel, stainless steel, nickel, palladium, cobalt or alloys thereof. These materials prove more cost-effective for the production of the membrane than is the case, for example, for silicon, silicon dioxide or silicon nitride.

Aerosolizers with different membrane passage/hole geometries were tested, examples being funnel-shaped holes. Very good droplet formation was shown in particular by cylindrical and conical shaped passages. The techniques (e.g., lithography, laser cutting or the like) by which these holes are made in the membrane are familiar to the skilled person.

### FIGURES

FIG. 1 shows a graph of the viscosity of the antisense molecule-containing composition as a function of its concentration.

FIG. 2 shows results of viscosity measurements on the antisense molecule-containing composition as a function of its concentration.

FIG. 3 shows measurement results of an FAT (factory acceptance test) of compositions with different antisense molecule concentrations of the antisense molecule-containing composition.

FIG. 4 shows measurement results of an FAT of the antisense molecule-containing composition with antisense molecule concentrations of 20 mg/ml and 50 mg/ml.

FIG. 5 shows measurement results of the antisense molecule-containing composition which were by various aerosolizers with membranes having different perforations.

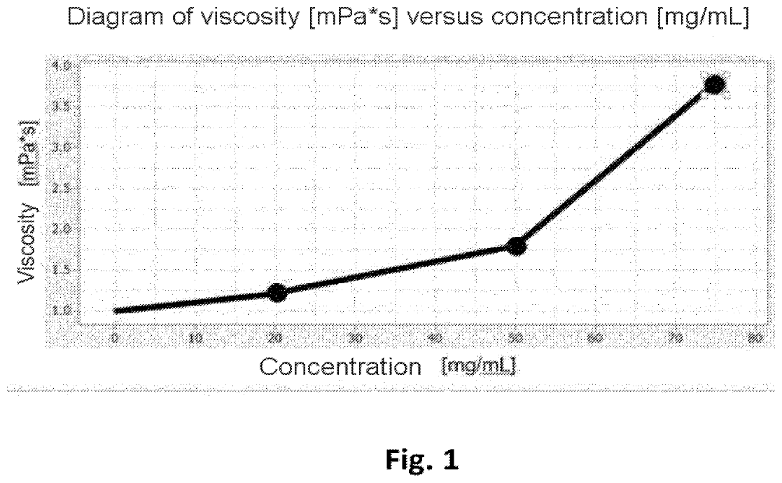

FIG. 1 shows a graph of the viscosity of an antisense molecule-containing composition as a function of its concentration. In the example shown, the antisense molecule is a DNAzyme. This DNAzyme can specifically downregulate the expression of GATA-3 and/or the expression of Tbet. In a preferred version, antisense molecules in particular from a group encompassing the DNAzymes hdg1 to hdg70 or from a group encompassing the DNAzymes td1 to td78 are provided. Preference is given to using the DNAzyme hgd40 with the sequence GTGGATGGAggctagctacaacgaGTCTTGGAG. Hdg40 specifically inhibits the expression of GATA-3. FIG. 1 shows a graph, illustratively, of the viscosity of an hgd40-containing composition. Along the Y axis of the graph from FIG. 1, the viscosity of the composition is plotted in mPa·s; the X axis indicates the DNAzyme concentration of the composition in mg/ml. From the plot it is apparent that the viscosity of the DNAzyme-containing composition undergoes a substantially exponential increase. A sharp increase in the viscosity can be observed in particular at a concentration of 50 mg/ml and higher. A viscosity of 3.5 mPa·s is observable even at a concentration of about 75 mg/ml.

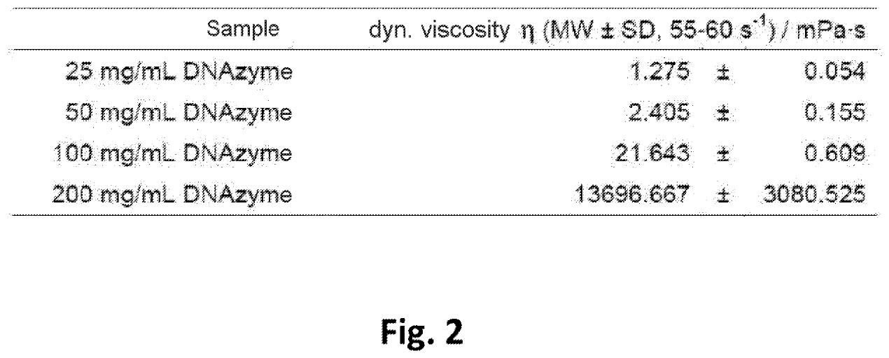

FIG. 2 shows the results of a viscosity measurement on an antisense molecule-containing composition as a function of its concentration, with the antisense molecules studied being DNAzymes. The results set out as a table demonstrate the exponential increase in the viscosity of an antisense molecule-containing composition having an antisense molecule concentration of between 50 mg/ml and 100 mg/ml. The correlation shown here between nucleic acid concentration and viscosity is not so pronounced for different, double-stranded DNA molecules; the viscosity of a double-stranded DNA-containing solution typically sees a substantially linear increase with increasing DNA concentration. At a viscosity of above 3.5 mPa·s, the pressure which an aerosolizer would have to achieve is too high. This may result in the passages or holes in the membrane becoming clogged or fouled. This risk is significantly minimized by a composition whose antisense molecule concentration is lower than 75 mg/ml, because the viscosity of such an antisense molecule-containing composition is below 3.5 mPa·s.

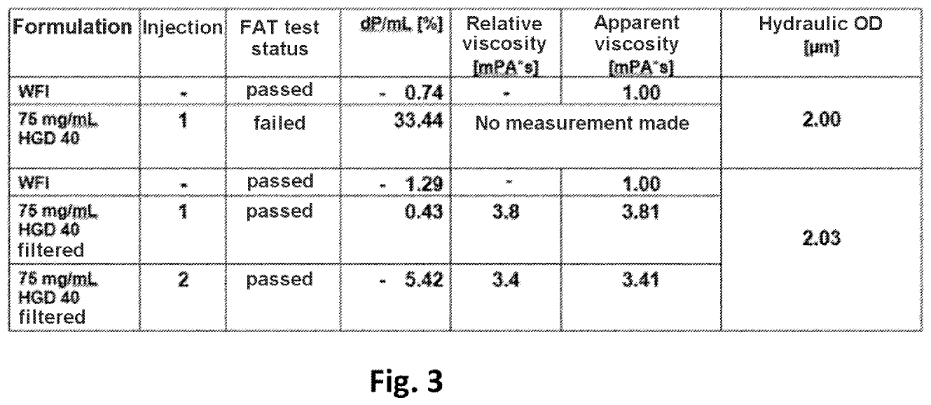

FIG. 3 shows in table form measurement results of an FAT (factory acceptance test) of compositions having different antisense molecule concentrations, with the antisense molecules being DNAzymes, specifically the DNAzyme hgd40. The purpose of an FAT is to test the usability of a product. The table shows that a composition having an antisense molecule concentration of above 75 mg/ml can no longer be misted by the aerosolizer from the test (cf. line 3 of the table from FIG. 3). If a composition having an antisense molecule concentration of 75 mg/ml is subjected to sterile filtration in advance of the test, the antisense molecule concentration is lowered to below 75 mg/ml. A composition having a DNAzyme concentration of this kind can be misted using the aerosolizer employed in the test (cf. lines 5 and 6 of the table from FIG. 3). The FAT reports the viscosity (dP/ml) in percent. In order to pass the FAT carried out here, the viscosity had to be below 5%. The abbreviation “WFI” in the second and fourth lines of the table from FIG. 3 stands for “Water for Injection” and represents the test control—accordingly, the “WFI” controls correspond to a viscosity of 1 dP/ml.

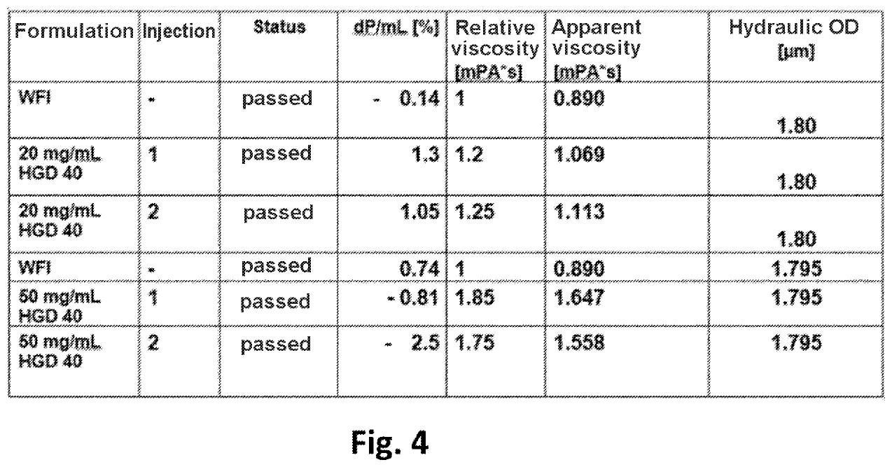

FIG. 4 shows measurement results of an FAT on a composition having antisense molecule concentrations of 20 mg/ml and 50 mg/ml, respectively. The antisense molecules studied here comprise, in particular, DNAzymes. The results show that such compositions pass the FAT and can be misted by means of the use according to the invention (cf. “passed” in lines 3, 4, 6 and 7 of the table from FIG. 4).

The measurement results from FIGS. 3 and 4 therefore show that an antisense molecule-containing composition, and more particularly a DNAzyme-containing composition having a DNAzyme concentration of below 75 mg/ml, can be used as an aerosol by means of an aerosolizer in accordance with the features presently described. Hence the use according to the invention is ideally suited to the treatment of a patient suffering from a respiratory tract disease associated with chronic inflammation. Preference here is given to using antisense molecule or DNAzyme concentrations of 20 mg/ml to 50 mg/ml.

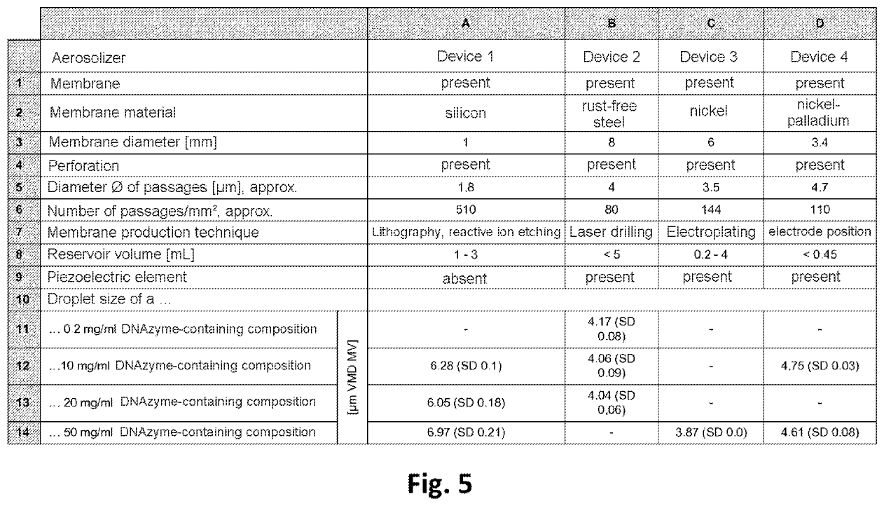

FIG. 5 shows measurement results on some of the aerosolizers tested. The measurement results show that aerosolizers having the features of the invention are ideally suited to generating an aerosol of an antisense molecule-containing composition (FIG. 5, columns B-D). In the example shown in FIG. 5, the antisense molecules are DNAzymes. In the case of the devices from columns B-D, the aerosol is formed by bringing the composition into contact with the aerosolizer membrane which has been set in vibration. In the invention this membrane has a perforation which is formed by at least one group of passages, the passages at their narrowest point having a diameter which is between 3 μm and 5 μm (FIG. 5, cells B5, C5 and D5), with the group of passages comprising at maximum 500 passages per mm² of the membrane (FIG. 5, cells B6, C6 and D6). In this case, droplets or particles having a size of around 4 μm are produced (FIG. 5, cells B11-B13, C14, D12 and D14), this being regarded as ideal for the generation of an aerosol of an antisense molecule-containing composition for the treatment of a patient suffering from a respiratory tract disease associated with chronic inflammation. The vibration of the membrane boosts the formation of droplets having the preferred size.

A device in which the passages at their narrowest point have a diameter of less than 3 μm (FIG. 5, cell 5A) or whose membrane has more than 500 passages per mm² (FIG. 5, cell 6A) results in an aerosol with droplets having a size of more than 5 μm VMD MV (FIGS. 12A, 13A and 14A). Droplets of this size have the drawback that because of impactation forces, they do not reach their corresponding site of action, or at least do so only to a greatly reduced extent. Accordingly, the antisense molecules and in particular the DNAzymes would not be able to develop their therapeutic effect at the site at which the chronic inflammation is present. One of the reasons for this is the hole density: with a hole density of above 500 passages per mm² of the membrane, it is possible for the resultant droplets or particles to undergo fusion, aggregation or combination to form larger particles directly after emergence from the passages.

1. A method for generating an aerosol of a composition, for administering the composition by inhalation,
where the composition comprises at least one antisense molecule,
where the aerosolizer has a membrane with a perforation,
where the composition, for generating the aerosol, is contacted with the membrane, and the membrane of the aerosolizer is set in vibration,
where the perforation is formed by at least one group of passages, and
where each passage of a group has a diameter at its narrowest point of at least 3 μm and at most 5 μm and where the at least one group of passages comprises at maximum 500 passages per mm² of the membrane.

2. The method of claim 1, characterized in that the membrane has a piezoelectric element which sets the membrane in vibration.

3. The method of claim 1, characterized in that the passages of the membrane are cylindrical.

4. The method of claim 1, characterized in that the passages of the perforation are conical, with the diameter of each conical passage decreasing in the direction of the region of the perforation at which the aerosol emerges from the perforation.

5. The method of claim 1, characterized in that the perforation of the membrane has at least two groups of passages.

6. The method of claim 1, characterized in that the membrane has a diameter of 6 mm to 8 mm.

7. The method of claim 1, characterized in that the membrane is produced from a material which comprises at least one metal, the metal being more particularly an element from the group of stainless steel, nickel, palladium, cobalt, rust-free steel and/or being an alloy of at least two of the elements.

8. The method of claim 1, characterized in that the composition has an antisense molecule concentration of below 75 mg/ml.

9. The method of claim 8, characterized in that the composition has an antisense molecule concentration of 20 mg/ml to 50 mg/ml.

10. The method of claim 1, characterized in that the composition has a viscosity which is lower than 3.5 mPa·s.

11. The method of claim 1, characterized in that the group of passages comprises at maximum 350 passages per mm² of the membrane.

12. The method of claim 1, characterized in that the group of passages comprises at maximum 200 passages per mm² of the membrane.

13. The method of claim 1, characterized in that the group of passages comprises at maximum 50 passages per mm² of the membrane.

14. The method of claim 1, characterized in that the composition comprises at least one antisense molecule which is selected from the group encompassing DNAzymes, siRNAs, asDNAs or Ribozymes.

15. The method of claim 14, characterized in that the composition comprises at least one antisense molecule which specifically downregulates the expression of GATA-3, or comprises at least one antisense molecule which specifically downregulates the expression of Tbet, where the antisense molecule is selected from a group encompassing the DNAzymes hdg1 to hdg70 or from a group encompassing the DNAzymes td1 to td78.

16. The method of claim 15, characterized in that the at least one antisense molecule is a DNAzyme which has the hdg40 sequence (GTGGATGGAggctagctacaacgaGTCTTGGAG).
